SEQUENCE LISTING (5 sequences)

SEQ ID NO: 1 - protein, 6 aa, artificial
  peptide 2d (J3)
DYGNHE

SEQ ID NO: 2 - protein, 6 aa, artificial
  peptide 1d
DKGNHE

SEQ ID NO: 3 - protein, 6 aa, artificial
  peptide J3.1
DYGNYE

SEQ ID NO: 4 - protein, 6 aa, artificial
  peptide J3.2
MYGNHE

SEQ ID NO: 5 - protein, 6 aa, artificial
  peptide J3.3
LYGNHE

(19) United States
(12) Patent Application Publication
(10) Pub. No.: US 2024/0082341 A1
(43) Pub. Date: Mar. 14, 2024

(54) PEPTIDE FOR USE IN THE TREATMENT OR PREVENTION OF COVID-19

(71) Applicant: Instytut Biochemii I Biofizyki Polskiej Akademii Nauk, Warszawa (PL)

(72) Inventors: Norbert ODOLCZYK, Warszawa (PL); Piotr ZIELENKIEWICZ, Warszawa (PL); Maria WINIEWSKA-SZAJEWSKA, Podkowa Lesna (PL); Jaroslaw POZNANSKI, Warszawa (PL); Urszula ZIELENKIEWICZ, Warszawa (PL); Joanna KLIM, Prostki (PL)

(73) Assignee: Instytut Biochemii I Biofizyki Polskiej Akademii Nauk, Warszawa (PL)

(21) Appl. No.: 18/270,019

(22) PCT Filed: Dec. 28, 2021
(86) PCT No.: PCT/PL2021/050094

§ 371 Date: Jun. 28, 2023

(30) Foreign Application Priority Data
Dec. 28, 2020 (PL) P.436491

(51) Int. Cl. A61K 38/08; A61P 31/14

(52) U.S. Cl. CPC A61K 38/08; A61P 31/14

(57) ABSTRACT

Improved peptides suitable for blocking the interaction between SARS-COV-2 virus S glycoprotein and the human ACE2 receptor are disclosed, in particular for the treatment or to assist the treatment of an infection caused by SARS-CoV-2 virus and/or viruses with high genome sequence similarity to the SARS-CoV-2 virus.

The invention relates to peptides that are useful in blocking the interaction between the S glycoprotein of SARS-CoV-2 virus and the human ACE2 receptor, in particular for the treatment or to assist the treatment of an infection caused by SARS-CoV-2 virus and/or viruses with high genome sequence similarity to the SARS-CoV-2 virus.

### STATE OF THE ART

In December 2019, in China, atypical pneumonia caused by a new pathogen belonging to the coronavirus group was diagnosed for the first time¹. Due to the high genetic similarity to SARS-CoV (severe acute respiratory syndrome-coronavirus), the newly identified pathogen was named SARS-CoV-2², and the disease caused by it is COVID-19 (COronaVIrus Disease 2019)³.

Within less than two months, infections caused by the new virus were diagnosed on all continents, and the World Health Organization (WHO) declared a pandemic at the beginning of March⁴. According to WHO data, by mid-August 2020, over 2.0 million cases of COVID-19 and almost 740,000 fatalities were reported⁵, which in many countries led to overload and failure of healthcare systems⁶.

The most common disease symptoms of COVID-19 include: high temperature, cough, shortness of breath, muscle aches, headache, diarrhea, loss of taste and smell⁷. In the most serious cases, the virus causes widespread pneumonia and acute respiratory failure, requiring hospitalization in intensive care units and the use of mechanical breathing aids or even extracorporeal membrane oxygenation (ECMO)⁸.

Until recently, there had been no drugs known to directly combat the viral infection caused by SARS-CoV-2, and the treatment used was symptomatic. Currently, the only pharmaceutical preparation that seems to block the development of the SARS-CoV-2 virus is the broad-spectrum antiviral drug Remdesivir (Veklury®), which has been conditionally approved by the US Food and Drug Administration (FDA) and the European Medicines Agency (EMA)⁹.

In addition, many research centers and pharmaceutical companies have taken up the challenge of developing a vaccine against the SARS-CoV-2 virus, which appears to be the most effective way to combat viral transmission¹⁰, and some of these studies have entered Phase 3 clinical trials (NCT04470427)¹¹. Unfortunately, due to previous unsuccessful attempts to develop a vaccine against viruses closely related to SARS-CoV-2, such as SARS-CoV or MERS (Middle East Respiratory Syndrome), the quick achievement of an effective vaccine is questionable¹⁰.

The viral genetic material is single-stranded, positive-sense ribonucleic acid ss-RNA(+) surrounded by a nucleocapsid and a protein-lipid membrane on the surface of which there are viral proteins involved in the process of infecting host cells by the viral particles¹².

One of the key viral proteins involved in the infection process is the S (spike) glycoprotein, which binds directly to the host cell membrane receptor, triggering the entire cascade of events leading to the release and multiplication of the virus genetic material inside the cells of the infected organism¹³.

Two functional subunits can be distinguished in the S glycoprotein: S1 containing the receptor binding domain (RBD), which directly interacts with the host cell receptor, and S2, which participates in the fusion of the viral envelope with the host cell membrane. As demonstrated for SANS-CoV-2 virus, the membrane receptor is angiotensin-converting enzyme 2 (ACE2)¹³.

From the perspective of a possible antiviral therapy, it seems justified to consider the interaction between the human ACE2 receptor and the S1 subunit of the S glycoprotein as a potential therapeutic target in combating viral infection. Inhibiting such an interaction with an inhibitor of protein-protein interactions may lead to a reduction in the number of infected cells and mitigate or even block the development of viral infection in the human body.

Peptides of a sequence that corresponds with the interaction interface regions of one of the partners within the complex formed, seem to be a convenient starting point for the development of such inhibitors of protein-protein interactions¹⁴.

### TECHNICAL PROBLEM

The object of the present invention is to provide substances suitable for blocking protein-protein interactions between the S1 subunit of viral S (spike) glycoprotein and the human hACE2 (human angiotensin converting enzyme type 2) receptor, which can be used to produce a therapeutic agent and/or an adjunct agent for the treatment of a viral infection caused by SARS-CoV-2 virus or viruses from the group of coronaviruses with high sequence similarity to the genome of SARs-CoV-virus.

A particular object of the invention is to identify improved derivatives of the peptides disclosed in patent application P.435261, which derivatives would have a higher affinity for the RBD domain of the S protein of the SARS-CoV-2 virus.

Unexpectedly, the problem thus defined has been solved in the present invention.

### THE ESSENCE OF THE INVENTION

The subject of the invention is a peptide containing the amino acid sequence of the formula:

A1 A2 X X A3 X

where:
A1 is Met, Trp, Phe, Leu, Ile, Ser, Thr or Asp, preferably Met, Trp, Phe or Asp,
A2 is Leu, Ile, Tyr, Met, Phe or Lys, preferably Tyr, Met or Phe or Lys, more preferably Tyr, Met or Phe.
A3 is Phe, Tyr, Trp, Lys, Arg, Asn, Gln or His, preferably Phe, Tyr, Trp or His, and
X is a protein amino acid or its known derivative,
   or a pharmaceutically acceptable derivative thereof selected from the group of compounds containing its salts or complexes.

Preferably, the peptide of the invention has the sequence shown as Seq. Id. No. 1 or 2 or 3 or 4 or 5.

A protein amino acid is understood to mean any amino acid found in proteins of natural origin, in particular selected from: Ala, Ile, Arg, Leu, Asn, Lys, Asp, Met, Phe, Cys, Pro, Gln, Ser, Glu, Thr, Trp, Gly, Tyr, His or Val. Other known amino acids, not found in proteins of natural origin, may also be used to construct the peptide of the invention.

A known amino acid derivative is understood to mean any modified amino acid residue. Examples of such known modifications are those that occur naturally (post-translational) or by synthetic modifications such as (but not limited to) phosphorylation, glycosylation, hydroxylation, methylation, sulfonylation.

Another object of the invention is a peptide as defined above for pharmaceutical or diagnostic use, especially for use in the treatment or prevention of COVID-19.

Any pharmaceutically acceptable derivative of the peptide of the invention may also be used according to the invention, especially salts or complexes thereof.

### DESCRIPTION OF THE FIGURES

The attached figures allow for a better explanation of the essence of the invention.

FIG. 1 shows the fragment of the hACE2 protein: 30-DKFNHEAEDLFYQ-42 (blue) and the RBD domains of the S1 subunit of the viral S protein (green) of SANS-CoV-2. The key amino acid residues necessary to maintain interactions between the peptide/peptides and the viral S protein are described in the figure.

FIGS. 2-3 show the measurement results (at 22° C.) of the strength of the interaction of the tested peptide with the surface of the viral protein obtained using Microscale Thermophoresis (MST) in the presence of the RBD domain of the S1 subunit, viral S glycoprotein and peptides 1d (FIGS. 2) and 2d/J3 (FIG. 3), respectively. The blue lines show a model fitted globally to four independent experiments; the red lines limit the 95% confidence interval of the fitted relationships; the data represented by grey points were excluded from the analysis.

FIGS. 4-7 show the measurement results (at 25° C.) of the strength of the interaction of the tested peptide with the surface of the viral protein obtained using Microscale Thermophoresis (MST) in the presence of the RBD domain of the S1 subunit, viral S glycoprotein and peptides: 2d/J3 (FIG. 4), J3.1 (FIG. 5), J3.2 (FIG. 6), J3.3 (FIG. 7), respectively. The curves in solid line show a model fitted globally to four independent experiments; the curves in dashed lines limit the 95% confidence interval of the fitted relationships; the data represented by grey points were excluded from the analysis.

FIG. 8 shows the measurements results (at 25° C.) of the strength of the protein-protein interaction between the RBD domain and the hACE2 receptor using MST and the impact of the presence of J3 and J3.2 peptides on the strength of this interaction.

FIG. 9. Peptide blocking of the interaction of the RBD domain of the viral S glycoprotein with the hACE receptor (ELISA assay). The columns show the mean absorbance value of all replicates. All peptides (J3/J3.2/J3.3) were tested at concentrations of 2.5, 5 and 10 mM. Samples of −RBD/+-peptide and −RBD/−peptide were used as controls. In order to verify the repeatability of the results, the tests were carried out in duplicate, and the control—in triplicate.

FIG. 10. Interaction of the RBD domain of viral S protein with the hACE2 receptor in the presence of peptide J3 (ELISA assay). The columns show the mean absorbance value of all replicates. Peptide J3 was tested at concentrations of 5.0, 5.5, 6.0, 7.0, 7.5 and 10 mM, Samples of −RBD/−peptide and −RBD/−peptide were used as controls. In order to verify the repeatability of the results, the tests were carried out in duplicate, and the control—in triplicate.

Moreover, in order to better inderstand the essence of the invention, it is further described in the following examples.

### EXAMPLE 1. PREPARATION OF THE PEPTIDES OF THE INVENTION

For the purposes of the present invention, the available spatial structures of the complexes of the S glycoprotein oaf SARS-CoV-2 virus and the human hACE2 receptor, which were deposited in the Protein Data Bank (PDB, http://www.rcsb.org/)¹⁵, were analyzed. In the further development of the invention, the structure with the identifier PDBid: 6M0J¹⁶ was used, on the basis of which the interaction interface of both proteins was analyzed using the COCOMAPS¹⁷, Pymol and Chimera¹⁸ software. The analysis of interactions (including contact surface complementarity, hydrophobic properties, the possibility of hydrogen bonding and salt bridges) led to identification of the native fragment of the hACE2 protein: 30-DKFNHEAEDLFYQ-42, on the basis of which the invention presented in this application was developed.

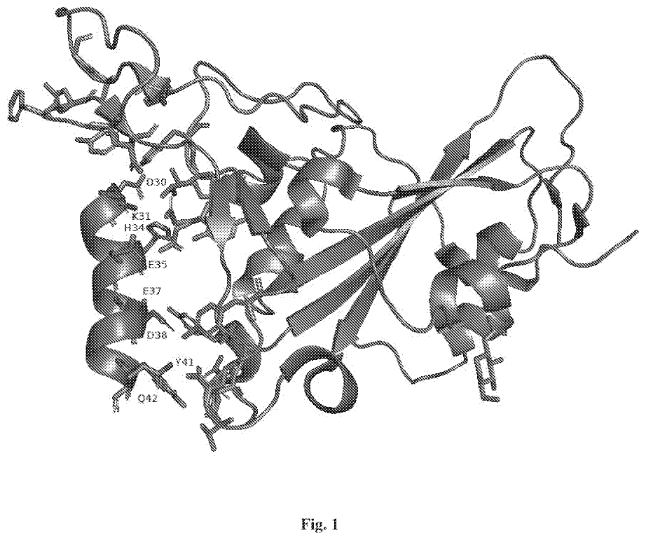

For the selected fragment of the hACE2 protein, amino acid residues were indicated, the occurrence of which in the sequence is crucial to maintain the desired interaction between the peptide/peptides and the viral S protein (FIG. 1).

For the purposes of the present invention, the interaction of the hexapeptide corresponding to the native fragment of the hACE2 protein: 30-DKFNHE-35, which, according to the patent application P.435261, has an affinity to the RBD domain of the S1 subunit of SARS-CoV-2 virus S glycoprotein, was analyzed. Detailed in silico analysis of the intermolecular interactions stabilizing the complex indicates the possibility of optimizing the hACE2 receptor sequence in terms of increasing the strength of interaction of such a motif with the molecular target.

Peptides of the sequence designed according to the invention may be obtained by any known peptide synthesis technique. In particular, they may be obtained by biotechnological methods or by chemical synthesis, e.g. by any solid-phase peptide synthesis (SPPS) method available in the art.

### EXAMPLE 2. BIOLOGICAL ACTIVITY OF THE PEPTIDES OF THE INVENTION

### Binding of the Peptides to the Surface of the RBD Domain of Viral S Glycoprotein (Microscale Thermophoresis)

In order to confirm the hypothesis that the proposed peptide inhibitors of protein-protein interactions can bind to the surface of viral S glycoprotein, experiments were performed using microscale thermophoresis (MST) in the presence of the RBD domain of the S1 subunit of viral S glycoprotein and the proposed peptide.

The protein concentration in all measurements was constant at 50 nM, while the peptide concentrations varied from 200 pM to 50 μM. All samples were prepared in 1×PBST (0.05%) and all measurements were carried out at 22° C. and/or 25° C.

The technique used allowed to estimate the dissociation constant (K_{D}) of the complex of peptide and the RBD domain of the SARS-CoV-2 S protein.

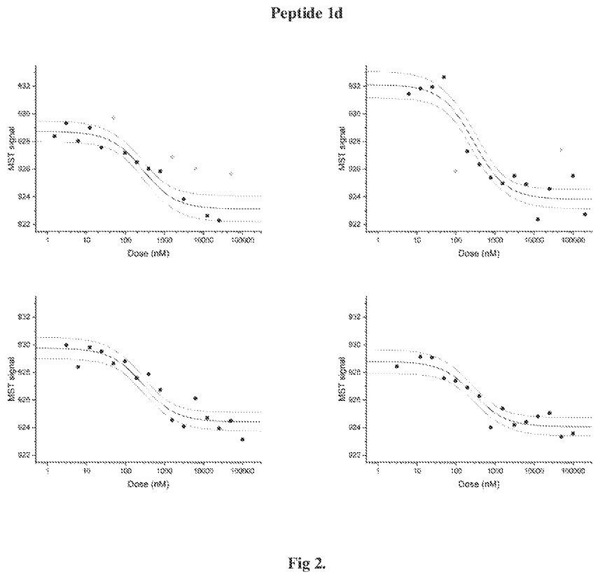

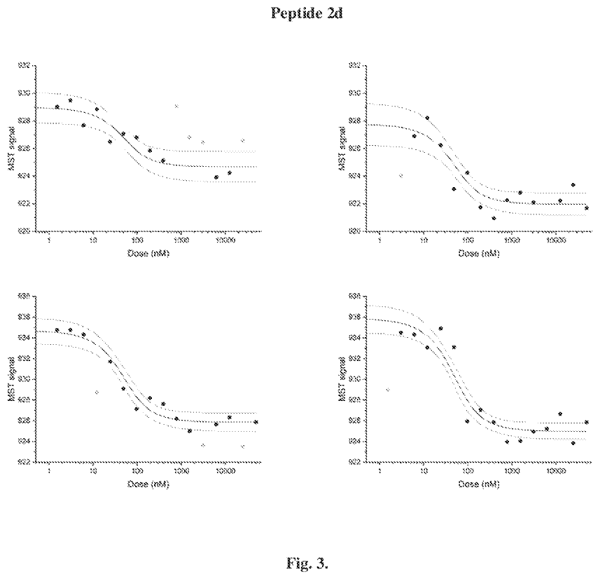

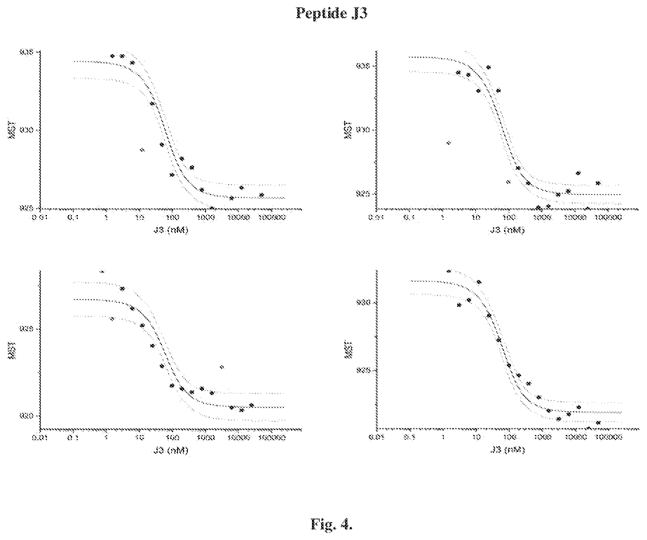

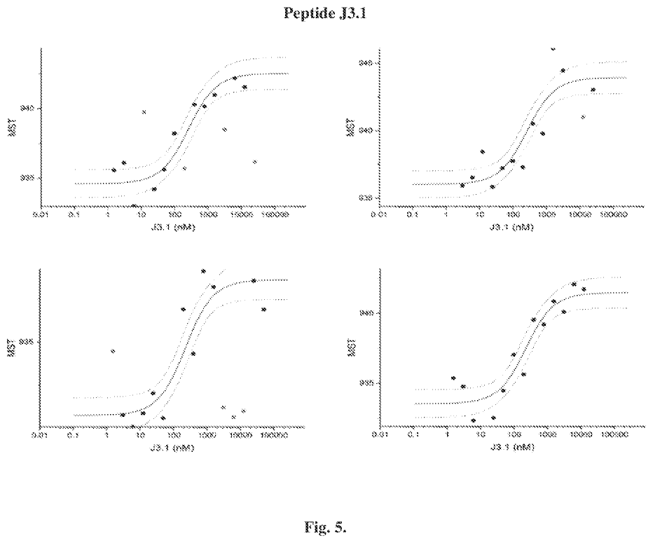

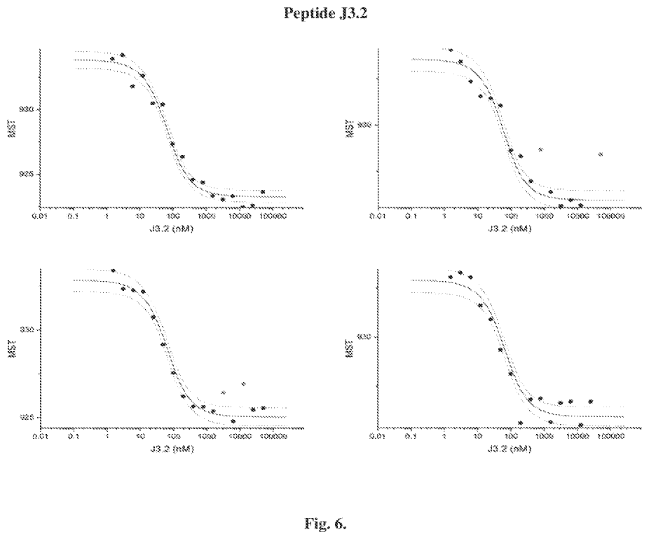

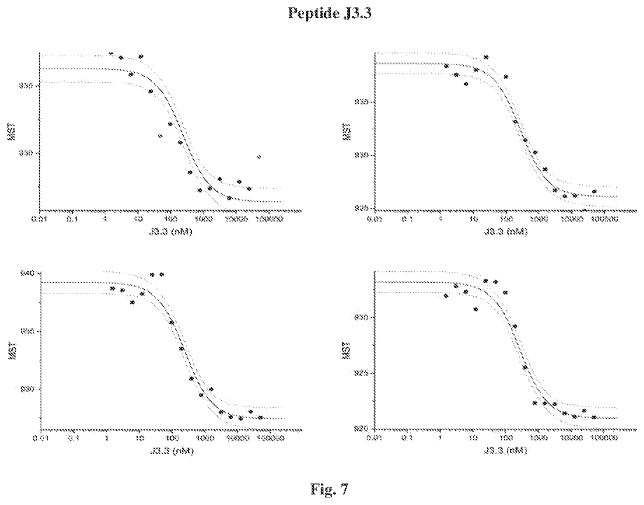

The binding strength for peptide of the sequence DYGNHE (J3) and MYGNHE (J3.2) is significantly higher than the native peptide DKCINHE (1d) proposed in the patent application P.435261, the results are presented in Table 1 and FIGS. 2-7.

TABLE 1
Dissociation constants of the (protein-
ligand) complex determined on the basis
of the MST experiments for the exemplary
peptides and the reference peptide
DKGNHE (1d).

| | | K_{D} (nM) | | K_{D} (nM) | |
|---|---|---|---|---|---|
| | | Tₘₑₐₛᵤᵣₑₘₑₙₜ | | Tₘₑₐₛᵤᵣₑₘₑₙₜ | |
| Id | Sequence | 25° C. | | 22° C. | |
| Peptide 1d | DKGNHE | | | 210 ± 50 | nM |
| Peptide 2d | DYGNHE | 35 ± 35 | nM | 23 ± 8 | nM |
| (J3) | | | | | |
| J3.1 | DYGNYE | 230 ± 75 | nM | | |
| J3.2 | MYGNHE | 33 ± 6 | nM | | |
| J3.3 | LYGNHE | 220 ± 35 | nM | | |
| J3.4 | MYGNYE | >10 | μM | | |
| 33.5 | LYGNYE | >1 | μM | | |

In a further step, in order to verify whether the proposed peptides, after binding to the surface of the viral protein, can block its interaction with the human ACE2 receptor, three-component experiments were performed in the system of: RBD domain of the S protein, hACE2 receptor and peptide.

### Peptide-Induced Blocking of the Interaction of the RBD Domain of Viral S Glycoprotein With the hACE2 Receptor (Microscale Thermophoresis)

First, two-component measurements were performed to estimate the dissociation constant K_{D} of the complex of the RBD domain of the S protein (fluorescently labelled) with the hACE2 protein. The concentration of the RBD domain was constant at 50 nM, and the concentrations of unlabelled hACE2 protein varied from 30 pM to 1 μM or 2.5 μM. The experiment was then repeated in the presence of the appropriate peptides at a constant concentration of 1 mM, observing the blocking of interaction between the proteins. All samples were prepared in 1×PBST (0.05%) and measurements were carried out at 25° C.

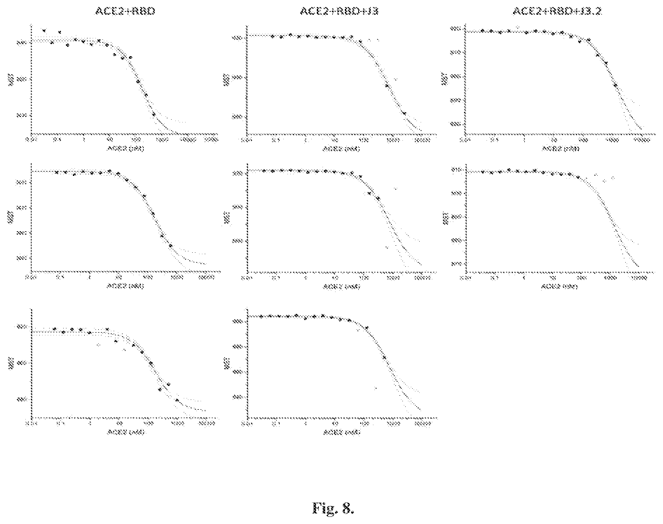

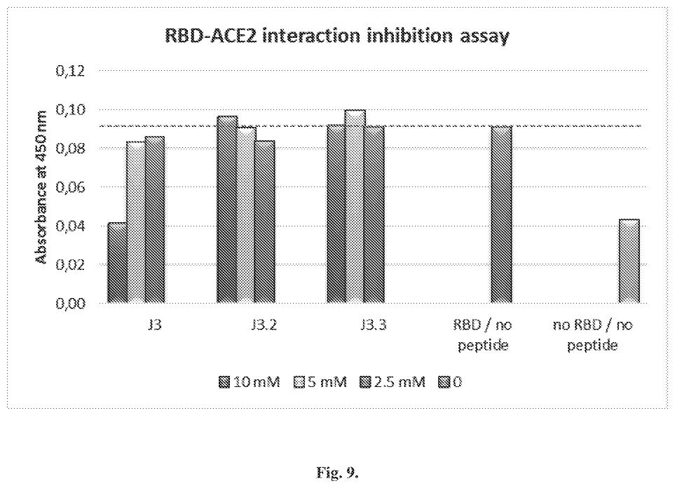

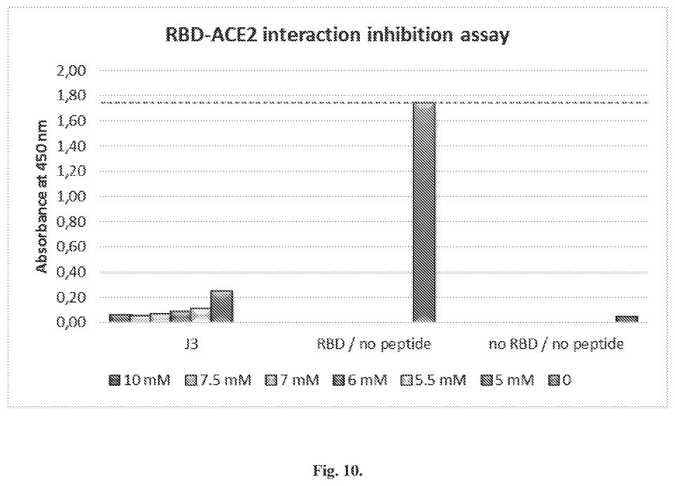

As shown in Table 2, the calculated dissociation constant K_{D} for the RBD-hACE2 interaction is ˜151 nM, while in the presence of the DYGNHE (J3) and MYGNHE (J3.2) peptides, it is significantly increased to ˜720 and ˜1370 nM, respectively (FIG. 8/Table 2).

TABLE 2
Dissociation constants of the RBD-hACE2
complex determined on the basis of the
MST experiments with no peptide and in
the presence of the DYGNHE (J3) and
MYGNHE (J3.2) peptides.

| | | K_{D} (nM) | |
|---|---|---|---|
| | | Tₘₑₐₛᵤᵣₑₘₑₙₜ | |
| Id | Sequence | 25° C. | |
| No peptide | — | 151 ± 23 | nM |
| J3 | DYGNHE | 720 ± 115 | nM |
| J3.2 | MYGNHE | 1370 ± 330 | nM |

The experiments used: (1) recombinant SARS-CoV-2 protein S1 subunit RBD domain (Arg319-Phe541) expressed in HEK293 cells (Human Embryonic Kidney 293) with the Monolith His-Tag Labeling Kit RED-tris-NTA 2^{nd} generation label attached to the C-terminal histidine tag according to the standard protocol provided by the manufacturer. The protein was provided by Genscript®/Raybiotech Inc. cat. no. 230-30162 (purity ≥95%); (2) recombinant extracellular domain (NP._068576.1) of hACE2 (Met1-Ser740) protein expressed in HEK293 cells with an hFc tag attached to the C-terminus; the protein was provided by SinoBiological. cat. no. SIN-10108-H02H (purity ≥95%). The MST experiment was performed with a Monolith NT 115 device (NanoTemper Technologies) using Premium MO-K025 capillaries (https://nanotempertech.com/).

The obtained experimental data were analyzed according to the method described earlier¹⁹, using appropriate models implemented in the OriginLab 2020B software (https://www.originlab.com/). The dissociation constant K_{D} was estimated using the so-called global fitting based on four independent MST pseudo-titration experiments using the temperature gradient-dependent thermal diffusion effect as an indicator of the protein-ligand interaction.

The analysis of the interaction interface between the proteins was performed using COCOMAPS¹⁷ software and the embedded software modules Pymol and Chimera¹⁸.

The analysis of the amino acid sequence change influence on the strength of interaction was performed using the FoldX programme. [doi: 10.1093/bioinformatics/btz184]

The DKGNHE (1d) peptide and the DYGNHE (2d/J3), DYGNYE (J3.1), MYGNHE (J3.2), LYONHE (J3.3), MYGNYE (J3.4), LYGNYE (J3.5) peptides were synthesized (purity ≥95%) and supplied by GenScript Biotech (Netherlands) B.V. (https://www.genscript.com/).

### Peptide-Induced Blocking of the Interaction of the RBD Domain of Viral S Glycoprotein With the hACE2 Receptor (ELISA Test)

Commercially-available “COVID-19 Spike-ACE2 binding assay kit II” (RayBiotech, USA, cat. no.: CoV-ACE2S2-2) in a 96-well plate version, the wells of which were coated with the ACE2 protein, was used for the ELISA test. In this assay, the total amount of ACE2 bound RBD protein (with Fc tag) is measured in the presence of the assayed peptides. In order to assess the amount of the RBD protein bound to ACE2, a colour reaction was performed using horseradish peroxidase enzyme conjugated with anti-Fc antibodies in the presence of 3,3′,5,5′-tetramethylbenzidine (TMB) substrate. The intensity of the colour reaction obtained was inversely proportional to the RBD protein concentration in the wells. The experiments were carried out according to the manufacturer's instructions. All incubations were at mom temperature with shaking. 100 μl of test compound (at various concentrations) mixed with the RBD protein was added to each well. The plate was then incubated for 2.5 hours. After a series of washes, 100 μl of horseradish peroxidase-conjugated anti-Fc antibody solution was added and incubated again. After another series of washes, 100 μl of TMB One-Step Substrate Reagent was added and incubated (protected from light) for another 30 min. The reaction was stopped by adding 50 μl of Stop Solution. Immediately after stopping the reaction, the absorbance was measured (at 450 nm) using a Varioskan Lux plate reader (ThermoFisher Scientific, USA).

### REFERENCES

1. Wang, C., Horby, P. W., Hayden, F. G. & Gao, G. F. A novel coronavirus outbreak of global health concern. The Lancet 395, 470-473 (2020).
2. Zhou, P. et al. A pneumonia outbreak associated with a new coronavirus of probable bat origin. Nature 579, 270-273 (2020).
3. Yan, R. et al. Structural basis for the recognition of SARS-CoV-2 by full-length human ACE2. Science 367, 1444-1448 (2020).
4. Timeline: WHO's COVID-19 response. https://www.who/int/emergencies/diseases/novel-coronavirus-2019/interactive-timeline.
5. WHO Coronavirus Disease (COVID-19) Dashboard. https://covid19.who.int.
6. Puca, E. et al. Short epidemiological overview of the current situation on COVID-19 pandemic in Southeast European (SEE) countries. J. Infect. Dev. Ctries. 14, 433-437 (2020).
7. Divani, A. A. et al. Coronavirus Disease 2019 and Stroke: Clinical Manifestations and Pathophysiological Insights. J. Stroke Cerebrovasc. Dis. 29, 104941 (2020).
8. Hazard, D. et al. Joint analysis of duration of ventilation, length of intensive care, and mortality of COVID-19 patients: a multistate approach. BMC Med. Res. Methodol. 20, 206 (2020).
9. Nabil, A. et al. Current coronavirus (SARS-CoV-2) epidemiological, diagnostic and therapeutic approaches: An updated review until June 2020. EXCLI J. 19, 992-1016 (2020).
10. Al-Kassiny, J., Pedersen, J. & Kobinger, G. Vaccine Candidates against Coronavirus Infections. Where Does COVID-19 Stand? Viruses 12, (2020).
11. A Study to Evaluate Efficacy, Safety, and Immunogenicity of mRNA-1273 Vaccine in Adults Aged 18 Years and Older to Prevent COVID-19—Full Text View—ClinicalTrials.gov. https://clinicaltrials.gov/ct2/show/NCT04470427.
12. Rabaan, A. A. et al. SARS-CoV-2, SARS-CoV, and MERS-COV: A comparative overview. Infez. Med. 28, 174-184 (2020).
13. Rossi, G. A., Sacco, O., Mancino, E., Cristiani, L. & Midulla, F. Differences and similarities between SARS-CoV and SARS-CoV-2: spike receptor-binding domain recognition and host cell infection with support of cellular serine proteases. Infection (2020) doi:10.1007/s15010-020-01486-5.
14. Fernández-Bachiller, M. I. et al. Mapping Protein-Protein Interactions of the Resistance-Related Bacterial Zeta Toxin-Epsilon Antitoxin Complex (ε2ζ2) with High Affinity Peptide Ligands Using Fluorescence Polarization. Toxins 8, 222 (2016).
15. Westbrook, J. et al. The Protein Data Bank: unifying the archive. Nucleic Acids Res. 30, 245-248 (2002).
16. Lan, J. et al. Structure of the SARS-CoV-2 spike receptor-binding domain hound to the ACE2 receptor. Nature 1-6 (2020) doi:10.1038/s41586-020-2180-5.
17. Vangone, A., Spinelli, R., Scarano, V., Cavallo, L. & Oliva, R. COCOMAPS: a web application to analyze and visualize contacts at the interface of biomolecular complexes. Bioinforma. Oxf. Engl. 27, 2915-6 (2011).
18. Pettersen, E. F. et al. UCSF Chimera—a visualization system for exploratory research and analysis. J. Comput. Chem. 25, 1605-1.612 (2004).
19. Winiewska, M., Bugajska, E. & Poznański, J. ITC-derived binding affinity may be biased due to titrant (nano)-aggregation. Binding of halogenated benzotriazoles to the catalytic domain of human protein kinase CK2. PLOS ONE 12, e0173260 (2017).

1. A peptide containing the amino acid sequence of the formula:
A1 A2 X X A3 X
where:
A1 is Met, Trp, Phe, Leu, He, Ser, Thr or Asp,
A2 is Tyr, Met, Phe, Leu, He or Lys, preferably Tyr, Met or Phe,
A3 is Phe, Tyr, Trp, Lys, Arg, Asn, Gin or His,
X is a protein amino acid or its known derivative,
or a pharmaceutically acceptable derivative thereof selected from the group of compounds containing its salts or complexes.

2. The peptide of claim 1, characterized in that it has a sequence selected from Seq. Id. No. 1-5.

3. The peptide of claim 1, characterized in that it has the sequence shown as Seq. Id. No. 5.

4. The peptide of claim 1 for pharmaceutical or diagnostic use, especially for use in the treatment or prevention of COVID-19.

5. The peptide of claim 1 for pharmaceutical or diagnostic use, especially for use in the treatment or prevention of infections caused by SARS-CoV-2 virus or viruses with high sequence similarity to the SARS-CoV-2 virus genome.

6. The peptide of claim 2 for pharmaceutical or diagnostic use, especially for use in the treatment or prevention of COVID-19.

7. The peptide of claim 3 for pharmaceutical or diagnostic use, especially for use in the treatment or prevention of COVID-19.

8. The peptide of claim 2 for pharmaceutical or diagnostic use, especially for use in the treatment or prevention of infections caused by SARS-CoV-2 virus or viruses with high sequence similarity to the SARS-CoV-2 virus genome.

9. The peptide of claim 3 for pharmaceutical or diagnostic use, especially for use in the treatment or prevention of infections caused by SARS-CoV-2 virus or viruses with high sequence similarity to the SARS-CoV-2 virus genome.
